(19) United States
(12) Patent Application Publication
(10) Pub. No.: US 2009/0171514 A1
(43) Pub. Date: Jul. 2, 2009

(54) METHOD FOR IMPROVING THE PRECISION AND RELIABILITY OF CIRCUIT HEATING CONTROL THROUGH A 1 - WIRE SENSOR

(75) Inventors: Honggang Yyang, Beijing (CN); Fei Cheng, Beijing (CN); Wei Wang, Beijing (CN)

Correspondence Address: SAWYER LAW GROUP LLP, 2465 E. Bayshore Road, Suite No. 406, PALO ALTO, CA, 94303, US

(73) Assignee: BEIJING AEONMED CO., LTD., Beijing (CN)

(21) Appl. No.: 12/344,485

(22) Filed: Dec. 27, 2008

(30) Foreign Application Priority Data
Dec. 28, 2007 (CN) 200710306310.6

(51) Int. Cl. G05D 23/00; A61M 16/00

(52) U.S. Cl. 700/300; 128/203.26

(57) ABSTRACT

A method for improving the precision and reliability of the heating control for an anesthetic machine circuit, comprises: a 1-wire temperature sensor is positioned in the anesthetic machine circuit and electrically connected to a control unit. The control unit reads the ID and verification code of the temperature sensor according to 1-wire protocol and determines the validity of the temperature sensor. The control unit reads the temperature value of the temperature sensor and compares it with the target control temperature. The control unit then turns on the control switch when requiring heating such that power supply powers the heater and the circuit temperature rises, or on the contrary turns off the control switch and the circuit temperature drops according to a control algorithm. The average power of the heater is controlled by PWM method such that the circuit is operated at a predetermined control temperature. With the usage of the 1-wire temperature sensor, the problem of analog signal transmission error is solved, the use of the sensor is facilitated, a long-distance temperature detection and control is obtained. At the same time the digital signal form and interface protocol of the sensor have relative higher anti-disturbance performance and failure recognition function. Accordingly, the requirement on the interface connection quality is reduced, and the reliability is improved.

Under 35 USC §120, this application claims the benefit of priority to CHINA Patent Application No. 200710306310.6, filed Dec. 28, 2007 entitled “A METHOD FOR IMPROVING THE REPCISION AND RELIABILITY OF CIRCUIT HEATING CONTROL THROUGH A 1-WIRE SENSOR”, all of which is incorporated herein by reference.

### FIELD OF THE INVENTION

The present invention relates to a method for improving the precision and reliability of the heating control for an anesthetic machine circuit, which is applicable for the heating control of devices involved in medical diagnosis, and more particularly for the temperature monitoring and control on the circuit heating part in an anesthetic machine or ventilator.

### BACKGROUND

At present, the anesthetic machine circuit is heated by the technical solution in which open-loop control or analog signal closed-loop control is employed. Usually, a heating element with a certain positive temperature factor, such as positive coefficient (PTC), is used to perform the heating. Meanwhile, the heating power is decreased automatically as the temperature is rising such that a certain heating temperature is reached. Such a technical solution has disadvantages of low precision control and poor reliability. Such a heating element has low temperature precision which is typically in the range of ±5 degrees. In a technical solution which uses the analog signal closed-loop control, a temperature sensitive element such as a thermistor is used to monitor and control the temperature, in which the precision is high, however there exist the following disadvantages: the anti-disturbance performance is poor; it requires high signal connection quality; the line compensation needs to be considered; the control circuit itself tends to be influenced by the temperature; the reliability is low; it is difficult to determine the sensor failure; and the overheating occurs easily.

Accordingly, what is needed is a system and method that overcomes the above-identified issues. The present invention addresses such a need.

### BRIEF SUMMARY

In order to improve the precision and reliability of the conventional control solution, one aspect of an embodiment provides a method for improving the precision and reliability of the heating control for an anesthetic machine circuit. The method, besides improving the precision and reliability of the heating control of an anesthetic machine circuit, may additionally reduce the requirements on the interfaces of the system, which provides for a long-distance heating control of the system. Another aspect of the embodiment invention provides a heating control system for an anesthetic machine circuit.

To achieve the above objectives, a 1-wire temperature sensor is adopted at the target position of the heating control, i.e., in the anesthetic machine circuit, and performs temperature measurement and control in cooperation with the heating element. With the 1-wire temperature sensor adopted, it is enough to connect temperature signal line, heating power supply output line, ground line (the heating power supply ground line may have the dual purpose as the sensor signal ground line). A microcontroller is used as the control unit. By using program, the sensor verification and the validity recognition are carried out based on 1-wire transmission protocol, and the function of temperature detection is fulfilled. According to certain algorithm such as PID, boundary restriction etc., the microcontroller controls average heating power by controlling the duty cycle of the heating output time, so as to control the temperature.

Depending on the type of the adopted controller, an embodiment may also include functions such as the input/output of data, condition indication, alarm and failure protection through I/O port or communication interface of the controller.

According to one aspect of an embodiment, a method for improving the precision and reliability of the heating control for an anesthetic machine circuit includes the steps of: A 1-wire sensor is positioned in the anesthetic machine circuit and the signals of the 1-wire temperature sensor are transmitted to the control unit; and a control unit processes the signals of the 1-wire temperature sensor so as to control the operation of a heating element.

According to another aspect of the present invention, a heating control system for an anesthetic machine circuit comprises: a control unit electrically connected to a control switch; a heating element used to heating the anesthetic machine circuit and electrically connected to a power supply via the control switch; a 1-wire temperature sensor which is positioned in the circuit and connected to the control unit via a temperature signal line, wherein the control unit receives the signal from the 1-wire temperature sensor and controls the average heating power by controlling the duty cycle of the heating output time.

When using a 1-wire temperature sensor, the precision is relative higher, which solves the problem of the analog signal transmission error. The use of the sensor is facilitated and a long-distance temperature detection and control is performed, and also the digital signal form and the interface protocol of the sensor have relative higher anti-disturbance performance and validity recognition function. Thus the requirements on the interface connection quality is reduced, and the reliability is improved.

It should be noted that the above description and the following detailed explanation is illustrative, which is intended to further describe the claimed invention.

A part from the above mentioned objectives, features and advantages, the present invention has other objectives, features and advantages which are hereinafter further described in details in conjunction with the accompanying drawings.

### BRIEF DESCRIPTION OF THE DRAWINGS

The figure included for constituting a part of the specification and further explaining the present invention shows the preferable embodiment and serves for describing the principle of the invention along with the description.

FIG. 1 is a block diagram according to an embodiment of the present invention.

The purpose, advantages and features of the present invention will become more apparent by description with reference to the following accompanying drawing.

### DETAILED DESCRIPTION OF THE INVENTION

The present invention relates to a method for improving the precision and reliability of the heating control for an anesthetic machine circuit, which is applicable for the heating control of devices involved in medical diagnosis, and more particularly for the temperature monitoring and control on the circuit heating part in an anesthetic machine or ventilator.

Hereinafter, an embodiment is described in detail in conjunction with the figure.

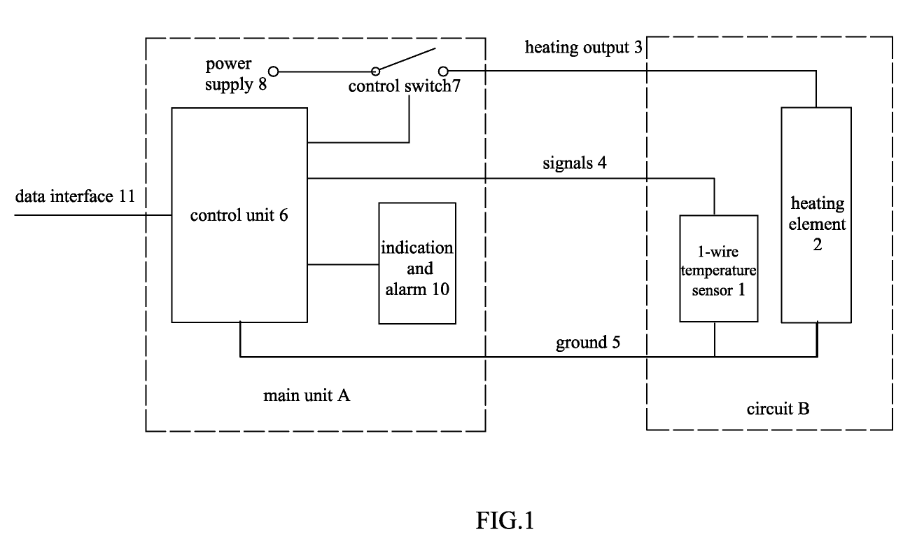

FIG. 1 is a block diagram of a system in accordance with an embodiment. The system includes a circuit B coupled to a main unit A. As shown, the circuit B is embodied by: a DS18B20 sensor in 1-wire series is selected as the temperature sensor 1 and a typical detecting point in the circuit is selected as the temperature measuring point of the temperature sensor 1. An electric heating element 2 may be embodied as a heating rod, heating wire or PTC element and so on, the heating power of which is selected based on the size and the heat dissipation of a circuit 3, so as to assure that the predetermined temperature is reached within the required preheating time. The main unit A includes a AT89C51, a PIC16F84 or other controllers which may be used as a control unit 6; a MOSFET or a relay may be used as a control switch 7. An indicator and alarm 10 may be realized by using the output pins of the controller to drive a LED or a buzzer. A data interface 11 may be realized by a serial interface of the control unit 6. The connection line between main unit A and the circuit B is very simple, that is, only the connection of the heating output 3, the signal 4 and the ground 5 is needed. Since digital signals are utilized, the signal line and the heating circuit may share the ground line.

Sampling program may be designed and realized according to 1-wire bus timing and command protocol (referring to the technical manual for DS18B20, http://www.maxim-ic.com.cn/pdfserv/en/ds/DS18B20.pdf). The temperature control program may employ a PID or a basic boundary restriction algorithm. When requiring heating, the control switch is turned on such that the heating element is powered to perform the heating. Whereas when requiring no heating, the control switch is turned off such that the heating element is powered off to stop the heating. The control programs of the alarm, indication and data communication may be designed as needed.

The basic operation principle and procedure are as follows:

1. The control unit 6 reads ID and verification code of the temperature sensor 1 according to 1-wire protocol and determines the validity of the temperature sensor 1;

2. The control unit 6 reads the temperature value of the temperature sensor 1 and compares it with the target control temperature, and then turns on the control switch 7 when requiring heating such that the power supply 8 powers the heater 2 and the circuit temperature rises, and turns off the control switch 7 when requiring to stop heating such that the heater 2 stops heating and the circuit temperature drops according to the control algorithm. A PWM method is provided in which the average power of the heater is controlled by controlling the heating time per unit time, the circuit B is operated at a predetermined control temperature.

The above is merely the description of the preferable embodiment of the present invention and not used for limiting the present invention. As for those skilled in the art, various changes, variations and omissions can be made to the present invention. Within the spirit and principle of the present invention, any change, substitution or improvement etc. should be covered by the scope of the present invention.

What is claimed is:

1. A method for improving the precision and reliability of the heating control for an anesthetic machine circuit, characterized in that, the method comprises steps:
a) positioning a 1-wire temperature sensor in the anesthetic machine circuit and connecting the sensor electrically to a control unit; and
b) processing the signals from the 1-wire temperature sensor by the control unit so as to control the operation of a heating element.

2. The method according to claim 1, wherein the 1-wire sensor is a DS18B20 type temperature sensor.

3. The method according to claim 1, wherein the heating element is a heating rod, heating wire or PTC.

4. The method according to claim 1, wherein the control unit is an AT89C51 chip or a PIC16F84 chip.

5. The method according to claim 1, wherein the method further comprises step c) the control unit controls indication and alarm devices.

6. The method according to claim 5, wherein the indication and alarm devices includes a LED and a buzzer.

7. The method according to any one of claims 1, wherein the control unit controls the heating operation of the heating element by a control switch.

8. The method according to any one of claims 2, wherein the control unit controls the heating operation of the heating element by a control switch.

9. The method according to any one of claims 3, wherein the control unit controls the heating operation of the heating element by a control switch.

10. The method according to any one of claims 4, wherein the control unit controls the heating operation of the heating element by a control switch.

11. The method according to any one of claims 5, wherein the control unit controls the heating operation of the heating element by a control switch.

12. The method according to any one of claims 6, wherein the control unit controls the heating operation of the heating element by a control switch.

13. The method according to claim 7, wherein the control switch is a MOSFET or a relay.

14. The method according to claim 13, wherein the control unit controls the average heating power by controlling the duty cycle of heating output time.

15. An heating control system for an anesthetic machine circuit, comprising:
a control unit electrically connected to a control switch; and
a heating element used for heating the anesthetic machine circuit and electrically connected to a power supply via the control switch, characterized in that, the system further comprises:
a 1-wire temperature sensor positioned in the circuit and connected to the control unit through a temperature signal line,
wherein the control unit receives the signals from the 1-wire temperature sensor and controls the average heating power by controlling the duty cycle of heating output time.
